(19) United States
(12) Patent Application Publication
(10) Pub. No.: US 2021/0244528 A1
(43) Pub. Date: Aug. 12, 2021

(54) ARTIFICIAL CORNEA

(71) Applicants: W. L. Gore & Associates, Inc., newark, DE (US); The Johns Hopkins University, Baltimore, MD (US)

(72) Inventors: Gopalan V. Balaji, Kennett Square, PA (US); Thomas B. Schmiedel, Middletown, DE (US); Anuraag Singh, Hockessin, DE (US); Esen K. Akpek, Baltimore, MD (US)

(21) Appl. No.: 17/252,017

(22) PCT Filed: Jun. 14, 2019
(86) PCT No.: PCT/US2019/037296

§ 371 Date: Dec. 14, 2020

Related U.S. Application Data
(60) Provisional application No. 62/684,901, filed on Jun. 14, 2018.

(51) Int. Cl. A61F 2/14; A61F 2/00; A61L 27/16; A61B 3/16; C08F 214/26; C08F 216/14

(52) U.S. Cl. CPC A61F 2/142; A61F 2/0077; A61L 27/16; A61B 3/16; C08F 214/262; C08F 216/1408; A61L 2430/16; A61F 2002/0086; A61F 2002/0081; A61F 2240/001; A61F 2002/009; A61F 2250/0051; A61F 2230/0013; A61F 2220/0008

(57) ABSTRACT

Artificial corneas suitable for surgical implantation are provided. Embodiments of artificial corneas include an optical an optical element that includes a body having an anterior side and a posterior side, an annular flange extending about the body, the anterior side including an anterior optical surface and the posterior side of the body including a posterior optical surface, and a tissue integration skirt coupled to the optical element, the tissue integration skirt being configured to promote tissue ingrowth, the tissue integration skirt being coupled to the optical element such that at least a portion of a periphery of the annular flange defined between the anterior and posterior sides of the optical element is covered by the tissue integration skirt. Also described are methods for implanting an artificial cornea of the present disclosure, the methods including providing the artificial cornea, removing a section of corneal tissue from the patient's cornea to form a tissue bed of existing tissue to which the artificial cornea can be affixed, implanting the art the artificial cornea such that the posterior side of the artificial cornea is suspended above the interior of the eye, and mechanically affixing the implanted artificial cornea to the existing corneal tissue of the tissue bed.

### FIELD

The present disclosure relates generally to artificial corneas. Artificial corneas of the present disclosure are suitable for implantation as a corneal replacement.

### BACKGROUND

The cornea generally refracts and focuses light onto the retina and serves as a protective barrier for the intra-ocular components of the eye. The cornea is subject to a host of diseases, genetic disorders, and trauma that can cause opacity of what should otherwise be an optically transparent window to the retina.

Although surgical procedures exist to replace damaged or diseased corneas with live tissue corneas taken from donor eyes, donor corneas may not be available, the underlying condition of the damaged eye may be such that donor cornea failure or rejection is likely, and/or the patient's physiology may be such that a donor cornea failure or rejection is likely.

In cases where implantation of a donor cornea is not viable, implantation of an artificial cornea is a potential alternative treatment. A corneal prosthesis or keratoprosthesis is an artificial cornea that can be implanted in a patient's eye to replace part of or all of a damaged or diseased cornea. The primary challenges facing keratoprostheses have been biointegration complications and extrusion of the device from the eye. Other complications include infection, retroprosthetic membrane formation, inflammation, glaucoma, lack of mechanical durability and optical fouling.

A number of approaches to solving the issue of device rejection have been attempted. One approach involves a keratoprosthetic design having a core and skirt type construction. The core and skirt type devices generally have a non-porous optical core for visual restoration and a skirt for bio-integration with the eye tissue surrounding the skirt.

However, to date, conventional core and skirt type constructions have not exhibited optimal device anchoring and long-term optical patency. As such, an improved keratoprosthesis that can demonstrate long-term optical patency is desirable.

### SUMMARY

According to one example (“Example 1”), an artificial cornea comprises: an optical element comprising a body having an anterior side and a posterior side, an annular flange extending about the body, the anterior side including an anterior optical surface and the posterior side of the body including a posterior optical surface; and a tissue integration skirt coupled to the optical element, the tissue integration skirt being configured to promote tissue ingrowth, the tissue integration skirt being coupled to the optical element such that at least a portion of a periphery of the annular flange defined between the anterior and posterior sides of the optical element is covered by the tissue integration skirt.

According to another example (“Example 2”), further to Example 1, the annular flange includes a first flange component and a second flange component situated posterior to the first flange component, the first flange component defining a first anterior surface and a peripheral surface, the second flange component defining a second anterior surface offset from the first anterior surface by the peripheral surface.

According to another example (“Example 3”), further to Example 2, the tissue integration skirt is coupled to each of the first anterior surface, the peripheral surface, and the second anterior surface.

According to another example (“Example 4”), further to Example 1 or Example 3, the first and second anterior surfaces of the annular flange are nonparallel.

According to another example (“Example 5”), further to any of Examples 2-4, the annual flange has a nonuniform thickness.

According to another example (“Example 6”), further to any of Examples 2-5, the first flange component and the second flange component each extend about the body radially outwardly therefrom.

According to another example (“Example 7”), further to any of Examples 2-5, the second flange component extents more radially outwardly than the first flange component.

According to another example (“Example 8”), further to any of Examples 2-5, the second flange comprises at least one aperture configured to allow tissue to proliferate therethrough.

According to another example (“Example 9”), further Example 8, the at least one aperture is formed by micro drilling.

According to another example (“Example 10”), further to Example 8, the second flange comprises a material having a microstructure that forms the at least one aperture.

According to another example (“Example 11”), further to any of Examples 1-10, the posterior optical surface is offset from a posterior surface of the annular flange.

According to another example (“Example 12”), further to Example 11, the offset between the posterior optical surface and the posterior surface of the annular flange is configured as a barrier to help resist a proliferation of tissue across the posterior optical surface.

According to another example (“Example 13”), further to any of Examples 1-12, the posterior side of the body is free from coverage by the tissue integration skirt.

According to another example (“Example 14”), further to any of Examples 1-13, the tissue integration skirt covers a portion of the anterior side of the optical element.

According to one example (“Example 15”), an artificial cornea comprises: an optical element configured to resist tissue ingrowth, the optical element comprising a body having an anterior side and a posterior side, the anterior side including an anterior optical surface and the posterior side of the body including a posterior optical surface, an annular flange extending about the body, the annular flange including a first flange component and second flange component situated posterior to the first flange component such that a peripheral surface of the body is defined between the first and second flange components, the first flange component defining a posterior flange surface, the second flange component defining an anterior flange surface offset from the posterior flange surface by the peripheral surface, and a tissue integration skirt being configured to permit tissue ingrowth, the tissue integration skirt being coupled to the peripheral surface.

According to another example (“Example 16”), further to Example 15, the integration skirt is further coupled to the anterior flange surface, the posterior flange surface, or both the anterior flange surface and posterior flange surface.

According to another example (“Example 17”), further to any of Examples 1-16, the anterior optical surface is convex.

According to another example (“Example 18”), further to any of Examples 1-17, the posterior optical surface is concave.

According to another example (“Example 19”), further to any of Examples 1-18, the optical element comprises a fluoropolymer.

According to another example (“Example 20”), further to Example 19, the fluoropolymer has been treated to render it hydrophilic.

According to another example (“Example 21”), further to Example 20, the fluoropolymer is hydrophilic.

According to another example (“Example 22”), further to any of Examples 1-21, the optical element comprises a copolymer of tetrafluoroethylene (TFE) and perfluoromethyl vinyl ether (PMVE).

According to another example (“Example 23”), further to any of Examples 1-22, the artificial cornea is foldable.

According to another example (“Example 24”), further to any of Examples 1-23, the artificial cornea is configured such that an intra-ocular pressure of an eye can be measured in situ through ocular tonometry involving interactions with the artificial cornea.

According to another example (“Example 25”), further to Example 24, the artificial cornea is configured such that an intra-ocular pressure of an eye can be measured in situ by measuring a deformation response of a region of the eye where the artificial cornea interfaces with native corneal tissue when acted on directly by a force external to the eye.

According to another example (“Example 26”), further to Example 25, the external force is applied by a physical body contacting the measured interface region.

According to another example (“Example 27”), further to any of Examples 1-26, a refractive index of the artificial cornea is in a range of between 1.3 to 1.4.

According to another example (“Example 28”), further to any of Examples 1-27, the optical element is configured to resist tissue ingrowth.

According to another example (“Example 29”), further to any of Examples 1-28, the anterior optical surface is configured to permit tissue attachment thereto while resisting tissue ingrowth.

According to another example (“Example 30”), further to Example 29, the anterior optical surface includes a microstructure configured to permit tissue attachment to the anterior optical surface while resisting tissue ingrowth.

According to another example (“Example 31”), further to Example 29, the anterior optical surface is at least partially covered by a corneal epithelial growth layer, the corneal epithelial growth layer being configured to encourage and support formation and maintenance of an organized monolayer of corneal epithelial cells over the anterior optical surface.

According to another example (“Example 32”), further to any of Examples 1-31, the optical element is formed of a material having a microstructure that is configured to resist tissue ingrowth.

According to another example (“Example 33”), further to any of Examples 1-32, the optical element is coated with a material that is configured to resist tissue ingrowth.

According to another example (“Example 34”), further to any of Examples 1-33, the tissue integration skirt is formed of a material having a microstructure that is configured to permit tissue ingrowth.

According to one example (“Example 35”), a method of forming an artificial cornea includes: providing an optical element having an anterior side and a posterior side, an annular flange extending about the body, the posterior side of the body including a posterior optical surface, providing a tissue integration skirt, the tissue integration skirt being configured to promote tissue ingrowth, coupling the tissue integration skirt to the optical element such that a portion of a periphery of the annular flange defined between the anterior and posterior sides of the optical element is covered by the tissue integration skirt.

According to another example (“Example 36”), further to Example 35, the posterior optical surface is longitudinally offset from a posterior surface of the annular flange.

According to another example (“Example 37”), further to Example 35 or Example 36, the tissue integration skirt is further coupled to the optical element such that a portion of the anterior side of the optical element is covered by the tissue integration skirt.

According to another example (“Example 38”), further to any of Examples 35-37, the optical element is configured to resist tissue ingrowth, and wherein the anterior side of the of the optical element is configured to permit tissue attachment while resisting tissue ingrowth.

According to one example (“Example 39”), a method of implanting an artificial cornea includes: providing the artificial cornea of any one of claims 1-34; removing a section of corneal tissue from a patient's cornea to form a tissue bed of existing corneal tissue to which the artificial cornea can be affixed; implanting the artificial cornea such that the posterior side of the artificial cornea is suspended above the interior of the eye; and mechanically affixing the implanted artificial cornea to the existing corneal tissue of the tissue bed.

According to another example (“Example 40”), further to Example 39, removing a section of corneal tissue includes removing a full-thickness section of corneal tissue from the patient's cornea, and wherein implanting the artificial cornea includes implanting the artificial cornea such that the posterior side of the artificial cornea is unsupported by the existing corneal tissue of the tissue bed.

While multiple embodiments are disclosed, still other embodiments will become apparent to those skilled in the art from the following detailed description, which shows and describes illustrative examples. Accordingly, the drawings and detailed description are to be regarded as illustrative in nature and not restrictive.

### BRIEF DESCRIPTION OF THE DRAWINGS

The accompanying drawings are included to provide a further understanding of inventive embodiments of the disclosure and are incorporated in and constitute a part of this specification, illustrate examples, and together with the description serve to explain inventive principles of the disclosure.

FIG. 1 is an illustration of an artificial cornea construction according to some embodiments;

FIG. 2 is a rear perspective view of the artificial cornea construction of FIG. 1 according to some embodiments;

FIG. 3 is a top view of the artificial cornea construction of FIG. 1 according to some embodiments;

FIG. 4 is a cross section view of the artificial cornea construction of FIG. 1 taken along line 4-4 of FIG. 3 according to some embodiments;

FIG. 5 is the cross-section view of the artificial cornea of FIG. 4 with the tissue integration element removed according to some embodiments;

FIG. 6 is a cross-section view of an artificial cornea construction according to some embodiments;

FIG. 7 is an illustration of an artificial cornea according to some embodiments;

FIG. 8 is a rear perspective view of the artificial cornea construction of FIG. 7 according to some embodiments;

FIG. 9 is a top view of the artificial cornea construction of FIG. 7 according to some embodiments;

FIG. 10 is a cross section view of the artificial cornea construction of FIG. 7 taken along line 10-10 of FIG. 9 according to some embodiments;

FIG. 11 is the cross-section view of the artificial cornea of FIG. 10 with the tissue integration element removed according to some embodiments;

FIG. 12 is an illustration of an artificial cornea construction according to some embodiments;

FIG. 13 is the cross-section view of the artificial cornea of FIG. 12;

FIG. 14 is an illustration of an artificial cornea construction according to some embodiments;

FIG. 15 is a rear perspective view of the artificial cornea construction of FIG. 14 according to some embodiments;

FIG. 16 is a top view of the artificial cornea construction of FIG. 14 according to some embodiments;

FIGS. 17A-17C are cross section views of the artificial cornea construction of FIG. 14 taken along line 17-17 of FIG. 16 according to some embodiments;

FIG. 18 is a cross section view of the artificial cornea core of FIGS. 17A-17C with the tissue integration element removed according to some embodiments;

FIG. 19 is a cross section view of an artificial cornea construction according to some embodiments.

FIG. 20 is a graphical representation showing the relationship between a measure of diopter and intra-ocular pressure for the artificial cornea according to some embodiments.

### DETAILED DESCRIPTION

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

Various aspects of the present disclosure are directed toward artificial cornea devices, systems, and manufacturing and implantation methods. More specifically, the present disclosure relates to devices, systems, and methods for making and using an artificial cornea comprising a core and skirt construction. The artificial cornea 100 is an implantable medical device that operates as a synthetic replacement for diseased corneas, damaged corneas, or corneas otherwise requiring replacement. In various embodiments, the artificial cornea includes an optical element and a tissue integration element coupled to the optical element. In various embodiments, the optical element is synthetic and comprised of a polymeric material. In various embodiments, the tissue integration element is synthetic and comprised of a polymeric material. The tissue integration element is configured to facilitate bio-integration of the artificial cornea into the eye while the optical element operates as a functional replacement to the existing cornea.

In some embodiments, the tissue integration element is configured to permit tissue ingrowth and tissue attachment to the material of the tissue integration element. In some embodiments, one or more designated portions or regions of the optical element may be configured to resist tissue ingrowth and attachment. For instance, in some embodiments one or more of the optical surfaces of the optical element (e.g., a posterior optical surface) may be configured to resist tissue ingrowth and attachment. Additionally or alternatively, in some embodiments, one or more designated portions or regions of the optical element may be configured to permit tissue attachment while being configured to resist tissue ingrowth. That is, in some embodiments, one or more portions or regions of the material of the optical element may be configured to permit tissue attachment. For instance, in some embodiments an optical surface of the optical element (e.g., an anterior optical surface) may be configured to permit tissue attachment while being resistant to tissue ingrowth.

Tissue ingrowth can be generally understood to mean cellular penetration into a material beyond the surface of the material (e.g., material may include a base material and/or a coating). Tissue ingrowth is generally associated with the microstructure of the material including pores or voids of a size sufficient to allow biological cells to grow or otherwise advance through the pores or voids. Thus, tissue ingrowth means that tissue can grow not only across a surface of the material (and reside on a surface of the material), but that the tissue can also penetrate substantially into the material beyond the surface of the material. As used herein, the term “tissue attachment,” on the other hand, can be generally understood to mean cellular adhesion or attachment to a surface of the material, without cellular penetration into the material beyond the surface of the material or substantially beyond the surface of the material. Adherence may be due to surface charges, surface roughness and/or chemical bonding. For example, the material may have a textured surface that is not smooth and that includes peaks, valleys, ridges, and/or channels that can support tissue residing thereon and therein. In some examples, the microstructure of the material may be non-porous, while in other examples the microstructure may include pores or voids that are of an insufficient size to accommodate cellular advancement therethrough. Thus, while the surface is configured to support tissue residing thereon and growing therearcoss, the tissue cannot penetrate substantially into the material beyond the surface of the material (e.g., beyond peaks, valley, ridges, and/or channels). Permitting tissue attachment while resisting tissue ingrowth provides that tissue, such as epithelial tissue, can proliferate and grow across the surface of the material without penetrating substantially into the material. Avoiding substantial penetration of tissue into one or more regions or portions of the optical element helps minimize a potential for fouling the optical performance of the optical element, as tissue ingrowth may degrade or otherwise foul the optical performance of the material of the optical element. Moreover, minimizing a potential for tissue to penetrate substantially into one or more regions or portions the optical element provides that tissue adhering to the surface can be subsequently removed from the surface by a physician, whereas tissue that has penetrated substantially into the optical element is difficult to remove (if even at all possible). In some instances, tissue cells growing across the optical surface may become arranged in an unorganized manner that causes unsatisfactory distortion of an image when viewed through the optical element. In these instances, the cells may have to be periodically scraped off the surface of the optical element to which they are attached. Limiting the cells to attachment to the surface, and minimizing penetration into the material beyond the surface provides physicians the ability to remove the cells from the optical element, such as by way of scraping the cells off of the surface. In some embodiments, adherence of tissue to the optical surface helps convert or transform the mesoplant (e.g., a device interfacing between the external and internal environment) into an implant, thereby minimizing the risk of infection and device extrusion.

An artificial cornea 100 according to some embodiments is illustrated in FIG. 1. As shown, the artificial cornea 100 includes an optical element 200 and a tissue integration element 300 (also referred to as a tissue integration skirt). The artificial cornea 100 has an anterior side 102 and a posterior side 104 opposite the anterior side 102. When implanted the anterior side 102 generally faces or is otherwise exposed to an outside environment, while the posterior side 104 faces an interior of the native eye. Thus, when implanted, the artificial cornea 100 may form a barrier between the interior of the eye and the outside environment. The artificial cornea 100 may include a front profile corresponding with a generally circular, elliptical or ovular shape. One or more of the anterior or posterior optical surfaces (discussed in detail below) may be curved or non-curved, such that an edge profile of the artificial cornea may correspond with the anterior and posterior optical surfaces being curved or non-curved.

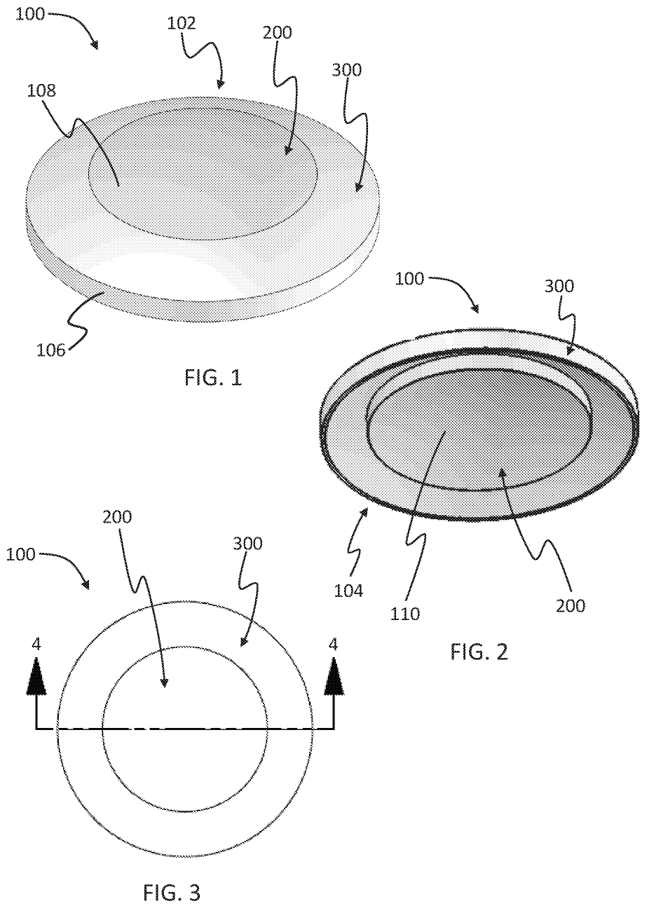

In some examples, an outer peripheral surface 106 of the artificial cornea 100 generally extending about a periphery of the artificial cornea of the artificial cornea 100 may be regularly or irregularly shaped (e.g., scalloped, spoked, star-shaped, etc.) and generally extends about a periphery of the artificial cornea. The artificial cornea 100 includes an anterior optical surface 108 and a posterior optical surface 110. As discussed in greater detail below, the anterior and posterior optical surfaces 108 and 110 of the artificial cornea 100 generally correspond to anterior and posterior optical surfaces of the optical element 200, and are thus shaped accordingly, as those of skill will appreciate. For example, as shown in FIGS. 1, 2, and 4, the anterior side 102 is generally convex and a posterior side 104 is generally concave.

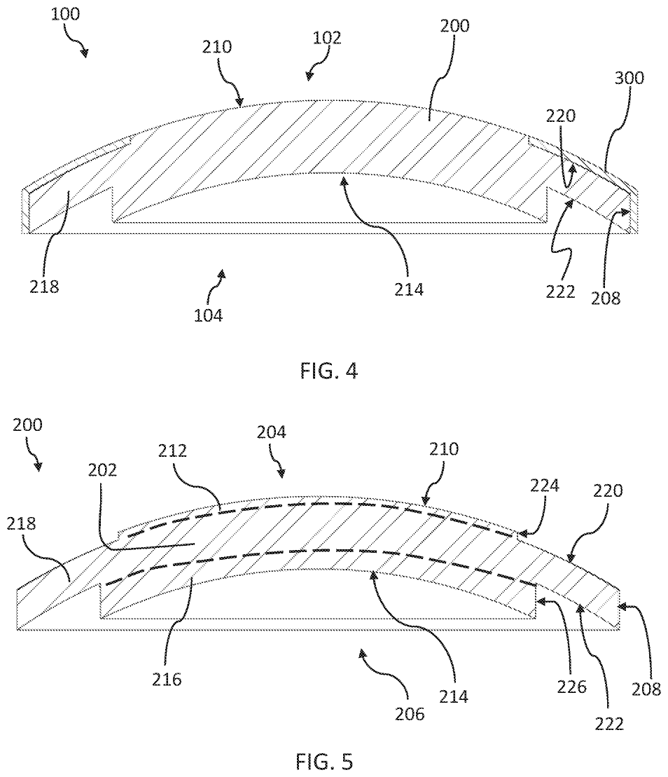

FIG. 4 shows a cross sectional view of an artificial cornea 100 taken along line 4-4 of FIG. 3. As shown, the artificial cornea includes an optical element 200 and a tissue integration element 300. The tissue integration element 300 is shown coupled to the optical element 200 along a peripheral wall or surface 208 thereof and along an anterior surface 220 thereof.

The optical element 200 shown in FIGS. 1-5 is a disc-shaped member that operates as an optically transparent window to the retina, when implanted in a patient's eye. FIG. 5 shows the optical element 200 with the tissue integration element removed. The optical element 200 generally includes a body 202, which may be disc-shaped as shown. Accordingly, it will be appreciated that the body 202 may include a circular or an elliptical shape, and may be flat or curved. In various embodiments, the body 202 is formed of a synthetic biocompatible material.

For instance, the body 202 may be formed from a number of suitable materials including, but not limited to, fluoropolymers selected from a copolymer of tetrafluoroethylene (TFE) and perfluoroalkyl vinyl ether (PAVE) such as perfluoromethyl vinyl ether (PMVE) perfluoroethyl vinyl ether (PEVE) and perfluoropropyl vinyl ether (PPVE), a copolymer of TFE and hexafluoropropylene (FEP), perfluoropolymers preferably containing TFE as a comonomer, perfluoroalkoxy polymer (PFA), perfluoropolyethers, or can comprise silicone, poly(methyl methacrylate) (PMMA), hydrogel, polyurethane, or any appropriate suitable combinations thereof.

In some examples, the body 202 may be formed from a material comprising a copolymer of TFE and PMVE, which is uniquely formed to have excellent mechanical properties while being substantially non-crosslinkable, i.e., free of cross-linking monomers and curing agents. The copolymer contains between 40 and 80 weight percent PMVE units and complementally between 60 and 20 weight percent TFE units. The lack of cross-linking systems ensures that the material is highly pure and, unlike some thermoset TFE/PMVE elastomers, is ideally suited as an implantable biomaterial. Advantages include excellent biocompatibility, high tensile strength, high clarity, high abrasion resistance, high purity, adequate elasticity, and ease of processing due to the thermoplastic and non-crosslinkable structure of the copolymer. The copolymer is thermoplastic and amorphous. It also is of high strength and can be used as a bonding agent particularly suited for bonding porous PTFE to itself or to other porous substances at room or elevated temperatures. It may also be used to bond nonporous materials including polymers such as nonporous PTFE. U.S. Pat. No. 7,049,380 further illustrates and describes such copolymers of TFE and PMVE and is herein incorporated by reference in its entirety.

In some embodiments, the body 202 is configured to minimize, inhibit, or even prevent tissue ingrowth. In some embodiments, a microstructure of the body 202 is configured to minimize, inhibit, or prevent tissue ingrowth. Additionally or alternatively, a coating applied to the body 202 is configured to minimize, inhibit, or prevent tissue ingrowth into the body 202. However, in some examples, tissue attachment to one or more of the surfaces of the body 202 (e.g., anterior optical surface 210) is permitted. In some examples, the anterior optical surface 210 may be configured to support tissue attachment while being resistant to tissue ingrowth (e.g., tissue penetration beyond the surface of the anterior optical surface 210 and into the material). In some embodiments one or more surface conditioning processes and/or material coating processes may be utilized to help promote tissue attachment to and proliferation across the anterior optical surface 210 of the optical element 200. For example, one or more known mechanical and/or chemical conditioning processes can be employed to condition the surface (e.g., condition the surface to have a non-smooth surface texture).

In some examples, the body 202 may have a refractive index in the range of 1.2 to 1.6, such as in the range of 1.3 to 1.4. In some examples, the body may have a light transmission in the visible light transmission range (wavelength of from 400-700 nm) of greater than 50%, more preferably greater than 80%. Additives such as cross-linking agents, biologically active substances (e.g., growth factors, cytokines, heparin, antibiotics or other drugs), hormones, ultraviolet absorbers, pigments, other therapeutic agents, etc., may be incorporated into the material forming the body 202 depending on the desired performance of the device.

In various embodiments, the body 202 is optically transparent in that the optical element 200 operates as a synthetic alternative to an otherwise normally functioning cornea. In some examples, one or more portions of the body 202, such as one or more optical portions, are optically transparent. For example, at least a portion of the body 202 situated interior to a coupling region between the optical element 200 and the tissue integration element 300 is optically transparent, as discussed in greater detail below.

In various embodiments, the body 202 of the optical element 200 includes an anterior side 204, a posterior side 206, and a peripheral surface 208 extending between anterior and posterior sides 204 and 206. In some embodiments, the anterior side 204 generally faces or is otherwise exposed to an outside environment, while the posterior side 206 faces the eye (e.g., eye tissue and eye interior). In various examples, the anterior side 204 is generally convexly curved, while the posterior side 206 is generally concavely curved. The peripheral surface 208 is a surface that circumferentially extends about the body 202 and forms a transition between the anterior and posterior sides 204 and 206. The peripheral surface 208 may be regular or irregular (e.g., scalloped), and may include one or more portions that extend normal or substantially normal to one or more surfaces of the anterior and posterior sides 204 and 206. The peripheral surface 208 may be linear or non-linear, and may be comprised of a plurality of surfaces (such as sub-surfaces) that collectively define the peripheral surface 208. The peripheral surface 208 generally forms or defines at least a portion of the coupling region where the tissue integration element 300 is coupled to the body 202. That is, in some embodiments, the tissue integration element 300 is coupled to the optical element 200 along a coupling region that is defined, at least in part, by the peripheral surface 208 (e.g., a portion of the body 202 having a surface that extends between the anterior and posterior sides 204 and 206).

In various examples, the anterior side 204 of the body 202 of optical element 200 includes an anterior optical surface, such as anterior optical surface 210. In various embodiments, the anterior optical surface 210 contributes to the formation of an image in the scope of visual acuity. The anterior optical surface 210 operates as the primary refractive surface in an optical path of light to the retina. In various examples, the anterior optical surface 210 operates as an interface between the body 202 of the optical element 200 of the artificial cornea 100 and the external environment, and defines at least a portion of the anterior side 102 of the artificial cornea 100 and at least a portion of the anterior side 204 of the body 202 of the optical element 200. The anterior optical surface 210 corresponds to the anterior optical surface 108 of the artificial cornea 100. In various examples, the anterior optical surface 210 is a surface capable of high light transmission. In various examples, the anterior optical surface 210 is generally curved or nonlinear. For example, as shown in FIG. 5, the anterior optical surface 210 is convex.

In some examples, the optical element 200 includes an anterior protrusion or a protrusion of the body 202 extending anteriorly from the body 202. For example, as shown in FIG. 5, the optical element 200 includes an anterior protrusion 212. The anterior protrusion 212 may be a protrusion of all of or less than all of the anterior side 204 of the body 202. Thus, as discussed further below and shown in FIG. 5, in various examples, the anterior side 204 of the body 202 may include a plurality of surfaces that are longitudinally offset from one another. In various examples, the anterior optical surface 210 corresponds to an anterior surface of the anterior protrusion 212. Thus, in examples including a plurality of anterior surfaces, the anterior optical surface 210 defines only a portion of the anterior side 204 of the body 202. However, in some other examples, the anterior optical surface 210 extends across an entire anterior side 204 of the body 202 and defines the anterior side 204 of the body 202.

In some embodiments, the anterior protrusion 212 is formed as a protrusion on the anterior side 204 of the body 202. In other examples, the anterior protrusion 212 is additionally or alternatively formed by forming an annular, peripherally extending recess in the anterior side 204 of the body 202. That is, in some examples, an annular ring of material is removed from the anterior side 204 of the body 202 to form an annular, peripherally extending recess about the anterior side 204 of the body 202. In yet other examples, the anterior protrusion 212 is additionally or alternatively formed by forming a peripherally extending annular flange 218 about the body 202, wherein an anterior surface 220 of the annular flange 218 is recessed or otherwise posteriorly offset relative to the anterior optical surface 210. Put differently, in some examples, the anterior side 204 of the optical element 200 is stepped such that it includes at least a first anterior surface and a second anterior surface that is offset relative to the first anterior surface. In some examples, the anterior optical surface is offset from the anterior surface 220 of the annular flange 218 in the range of between zero (0) and two-hundred (200) micron. As shown in FIG. 5, a first surface or step 224 extends between the anterior optical surface 210 and the anterior surface 220 of the annular flange 218.

In various embodiments, the posterior side 206 of the body 202 of optical element 200 includes a posterior optical surface, such as posterior optical surface 214. In various examples, the posterior optical surface 214 operates as an interface between the body 202 of the optical element 200 of the artificial cornea 100 and an interior of the eye, and defines at least a portion of the posterior side 104 of the artificial cornea 100 and at least a portion of the posterior side 206 of the body 202 of the optical element 200. The posterior optical surface 214 corresponds to the posterior optical surface 110 of the artificial cornea 100. In various examples, the posterior optical surface 214 is a surface capable of high light transmission. In some embodiments, the posterior optical surface 214 is free of surface defects or imperfections such as scratches, pits, or gouges. In various examples, the posterior optical surface 214 is generally curved or nonlinear. For example, as shown in FIG. 5, the posterior optical surface 214 is concave.

In some examples, the optical element 200 includes a posterior protrusion or a protrusion of the body 202 extending posteriorly from the body 202. For example, as shown in FIG. 5, the optical element 200 includes a posterior protrusion 216. The posterior protrusion 216 may be a protrusion of all of or less than all of the posterior side 206 of the body 202. Thus, as discussed further below and shown in FIG. 5, in various examples, the posterior side 206 of the body 202 may include protrusions that are longitudinally offset from one another. In various examples, the posterior optical surface 214 corresponds to a posterior surface of the posterior protrusion 216. Thus, in examples including a plurality of posterior surfaces, the posterior optical surface 214 defines only a portion of the posterior side 206 of the body 202. However, in some other examples, the posterior optical surface 214 extends across an entire posterior side 206 of the body 202 and defines the posterior side 206 of the body 202.

In some examples, the posterior protrusion 216 is formed as a protrusion on the posterior side 206 of the body 202. In other examples, the posterior protrusion 216 is additionally or alternatively formed by forming an annular, peripherally extending recess in the posterior side 206 of the body 202. That is, in some examples, an annular ring of material is removed from the posterior side 206 of the body 202 to form an annular, peripherally extending recess about the posterior side 206 of the body 202. In yet other examples, the posterior protrusion 216 is additionally or alternatively formed by forming a peripherally extending annular flange, such as annular flange 218, about the body 202, wherein a posterior surface of the annular flange is recessed or otherwise anteriorly offset relative to the posterior optical surface 214. Put differently, the posterior side 206 of the optical element 200 is optionally stepped (e.g., discontinuous) such that it includes at least a first posterior surface, and a second posterior surface that is offset relative to the first posterior surface. In some examples, the posterior optical surface 214 is offset from the posterior surface 222 of the annular flange 218 in a range of between zero (0) and one (1) millimeter.

In various examples, the anterior optical surface 210 is offset from the anterior surface 220 to facilitate placement and positional orientation and retention of the tissue integration skirt on the optical element 200. In some example, such an offset generally corresponds to a thickness of the tissue integration skirt, though such is not required. In various examples, the posterior optical surface 214 is offset from the posterior surface 222 to facilitate prevention of corneal tissue, which is situated about peripheral surface 208, from growing across the posterior side of the optical element and covering the posterior optical surface 214. In some instances, the presence of corneal tissue or other associated eye tissue on the posterior optical surface 214 may have a tendency to degrade or otherwise foul the optical performance of the optical element 200. In various examples, the posterior optical surface 214 may be offset from the posterior surface 222 by an amount that exceeds an expected thickness of abutting corneal tissue, which may be initially inflamed or swollen.

In some examples, an optical element that includes offset first and second posterior surfaces operates to further inhibit tissue ingrowth across the posterior side of the optical element. That is, in some examples, the step or surface extending between the first (e.g., optical) posterior surface and the second posterior surface operates to prevent a proliferation or propagation of tissue to the first posterior surface from the second posterior surface. For example, such a step operates as a barrier that helps prevent tissue growing across the second posterior surface (e.g., growing from a periphery of the optical element) from growing onto and across the first posterior surface. As shown in FIGS. 4 and 5, a surface or step 226 situated between the posterior optical surface 214 and the posterior surface 222 of the annular flange 218 operates to prevent or otherwise inhibit tissue proliferating from posterior surface 222 to posterior optical surface 214. Such a configuration operates to minimize or otherwise avoid fouling of the posterior optical surface 214 due to a presence of biological tissue. In various examples, the posterior optical surface 214 is treated with coatings to prevent tissue proliferation (e.g., including attachment and/or ingrowth) thereacross.

In some examples, the anterior and posterior protrusions 212 and 216 are similarly sized and/or shaped. For example, as shown in FIG. 5, the anterior and posterior protrusions 212 and 216 are generally circularly shaped. In some examples, the anterior and posterior protrusions 212 and 216 are dissimilarly sized and/or shaped. For example, as shown in FIG. 5, the posterior protrusion 216 protrudes more so from the body 202 than does the anterior protrusions 212. Likewise, as shown, the posterior protrusions 216 has a larger diameter than (or is otherwise more radially expansive than) the anterior protrusion 212.

In some examples, the anterior protrusion, a portion of the body, and a portion of the posterior protrusion form a core portion of the body 202 of the optical element 200. In some examples, the core portion of the body 202 is formed of a different material than is a remainder of the body 202 of the optical element 200. In some such examples, the core of the body 202 may be formed of an optically transparent and tissue ingrowth inhibiting material as described herein.

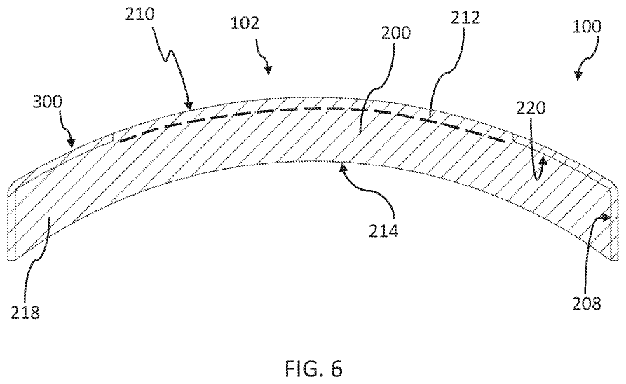

In certain embodiments, the posterior side 206 of the body 202 of optical element 200 does not include a posterior protrusion 216, as shown in FIG. 6.

As mentioned above, in various embodiments, the optical element 200 includes a peripheral annular flange (or flange portion), such as annular flange 218. In some embodiments, the annular flange 218 may be defined as that portion of the body 202 of the optical element extending radially outwardly of one or more of the anterior and posterior protrusions 212 and 216. In various embodiments, the annular flange 218 operates as both a region for coupling a tissue integration element 300 to the body 202 as well as an element through which one or more fastening elements can be passed (e.g., one or more sutures) for initially securing the artificial cornea 100 to the tissue of the eye, as discussed further below. In various examples, the annular flange 218 is defined by an anterior surface 220, a posterior surface 222, and a surface that extends between the anterior and posterior surfaces 220 and 222. In various examples, the surface that extends between the anterior and posterior surfaces 220 and 222 corresponds to the peripheral surface 208 of the body 202, mentioned above.

The peripheral surface 208 may including one or more portions that extend normal to or substantially normal to one or more of the anterior and posterior surfaces 220 and 222. Additionally or alternatively, the peripheral surface 208 may additionally or alternatively extend parallel to or substantially parallel to a central axis of the artificial cornea 100, and as mentioned above may be linear or non-linear, and may be comprised of a plurality of surfaces (such as sub-surfaces) that collectively define the peripheral surface 208. In some embodiments, the central axis of the artificial cornea 100 extends normal to one or more of the anterior and posterior optical surfaces 108 and 110 and intersects a central point or apex of one or more of the anterior and posterior optical surfaces 108 and 110. Thus, in some examples, the peripheral surface 208 extends normal to or substantially normal to an apex of one or more of the anterior and posterior optical surfaces 108 and 110 of the artificial cornea 100.

The optical element 200 may be formed through a compression molding process or other known processes. For instance, in some embodiments, the polymer material forming the optical element 200 is generally heated and compressed in a preformed mold that causes the heated polymer material to adopt the shape of the preformed mold, which closely resembles the desired shape of the optical element 200 as described herein. In some embodiments, after the optical element 200 is formed, the optical element 200 may be subjected to one or more finishing processes. For example, as discussed in greater detail below, the optical element 200 or the artificial cornea 100 may be subjected to one or more precision shaping processes wherein one or more of the associated optical surfaces are precision-shaped. Examples of other finishing processes include but are not limited to, post forming, surface smoothing (e.g., eliminating surface defects), polishing, wetting, trimming, and/or sterilization (such as chemical, heat, and/or steam sterilization).

Turning back again to FIG. 4, the artificial cornea 100 is shown with the tissue integration element 300 coupled to the annular flange 218. The tissue integration element 300 operates as a mechanical anchoring mechanism or element configured to facilitate a coupling of the artificial cornea 100 to the surrounding tissue of the eye. In some examples, the tissue integration element 300 is configured such that tissue can grow into and across the material of the tissue integration element 300, which helps maintain a position of the artificial cornea 100 within the eye.

In various examples, the tissue integration element 300 is microporous and configured to promote the ingrowth and attachment of surrounding tissue. In some examples, the tissue integration element 300 includes or is otherwise formed of one or more layers or sheets of a porous polymer material, such as expanded polytetrafluoroethylene (ePTFE). However, these layers or sheets may be formed from other polymers, including, but not limited to polyurethane, polysulfone, polyvinylidene fluorine (PVDF), polyhexafluoropropylene (PHFP), perfluoroalkoxy polymer (PFA), polyolefin, fluorinated ethylene propylene (FEP), acrylic copolymers, hydrogels, silicones and polytetrafluoroethylene (PTFE). These materials can be in sheet, knitted, woven, or non-woven porous forms. In some examples, the layers or sheets are laminated or otherwise mechanically coupled together, such as by way of heat treatment and/or adhesives and/or high-pressure compression and/or other laminating methods known by those of skill in the art

In some examples, the layers or sheets of polymer material forming the tissue integration element 300 or the tissue integration element 300 itself are subjected to one or more processes to modify the microstructure (and thus the material properties) of the layered polymer material. In some examples, such processes include but are not limited to, material coating processes, surface preconditioning processes, and/or perforation processes. Material coating processes may be utilized to apply one or more drug or antimicrobial coatings, such as metallic salts (e.g. silver carbonate) and/or organic compounds (e.g. chlorhexidine diacetate), to the polymer material. In some embodiments, material coating processes may be utilized to help promote tissue attachment to and proliferation across the tissue integration element 300 consistent with the discussion above. Hydrophilic coatings to enable immediate wetout of the polymer matrix can also be applied through one or more plasma treatment (or chemical modification wetting) processes, as generally polymer surfaces are hydrophobic in nature. For example, a surface of the polymer material may be modified with hydrophilic agents, thereby decreasing its hydrophobicity and improving its wettability. More specifically, the polymer material may be pre-treated with plasma to activate the surface, exposed to a hydrophilic polymer and treated again with plasma to crosslink the hydrophilic coating on the surface of the polymer material.

In some examples, one or more surface preconditioning processes may additionally or alternatively be utilized to form layers of the tissue integration element 300 exhibiting a preferred microstructure (e.g., wrinkles, folds, or other geometric out-of-plane or undulating structures), as explained in U.S. Patent Application Publication Number 2016/0167291, Ser. No. 14/907,668, filed Aug. 21, 2014, the entire contents of which are incorporated herein by reference. Likewise, one or more plasma treatments may be utilized to achieve a desired surface structure (e.g., stucco-like). Such surface preconditioning may facilitate a bolder early inflammatory phase after surgery, providing an early stable interface between the artificial cornea 100 and the eye tissue with which it interfaces. Additionally, in some examples, one or more perforation processes may additionally or alternatively be utilized to form a plurality of perforations or pores in the tissue integration element 300 which can further facilitate tissue ingrowth.

In some embodiments, one or more surface coatings comprising antioxidant components may be applied to one or more of the optical element 200 and tissue integration element 300 to mitigate the body's inflammatory response that naturally occurs during wound healing after surgery. Surfaces thereof can be modified with anti-proliferative compounds (e.g. Mitomycin C, 5-fluoracil) to moderate the surrounding tissue response in the eye.

In various examples, the polymer material of the tissue integration element 300 is subjected to the one or more processes to modify the microstructure prior to its application to the optical element 200. For example, the polymer material of the tissue integration element 300 may be subjected to a plasma treatment process to impart a surface structure on the material (e.g., for the promotion of tissue integration) prior to applying the polymer material to the optical element 200, as explained in U.S. Patent Application Publication Number 2006/0047311, Ser. No. 11/000,414, filed Nov. 29, 2014, the entire contents of which are incorporated herein by reference. In some examples, after treating the polymer material, the polymer material is sized and applied to the optical element 200. In some examples, the polymer material is cut to size, such as through one or more laser cutting or other suitable cutting processes as those of skill should appreciated.

It is to be appreciated that the tissue integration element 300 is coupled to the optical element 200 without compromising the optical performance of the optical element 200. That is, as discussed in greater detail below, the tissue integration element 300 is sized and shaped such that, when coupled to the optical element 200, the anterior and posterior optical surfaces 108 and 110 of the optical element 200 remain unobstructed by the tissue integration element 300. The tissue integration element 300 may thus be an annularly shaped member that, when coupled with the optical element 200, extends peripherally about one or more of the anterior and posterior optical surfaces 108 and 110.

As shown in FIG. 4, the tissue integration element 300 is coupled to the optical element 200 along the peripheral surface 208 and the anterior surface 220, without extending across the anterior and posterior optical surfaces 108 and 110, and without extending across the posterior surface 222. In some embodiments, the peripheral surface 208 thus forms or defines a first tissue integration element coupling region of the body 202. Similarly, in some embodiments, the anterior surface 220 of the annular flange 218 forms or defines a second tissue integration element 300 coupling region of the body 202.

Coupling the tissue integration element 300 to the optical element 200 along the periphery of the annular flange 218 as shown in the figures provides for tissue integration and device fixation along a peripheral surface of the artificial cornea 100 between the anterior and posterior sides 204 and 206 in a manner dissimilar to conventional devices and designs. Coupling the tissue integration element 300 to the optical element 200 such that the tissue integration element 300 extends along a portion of the anterior side 204 of the optical element 200 provides for tissue integration and device fixation partially along an anterior facing surface of the artificial cornea 100. Similarly, coupling the tissue integration element 300 to the optical element 200 such that the tissue integration element 300 extends along the peripheral surface 208 provides for tissue integration and device fixation along the periphery of the artificial cornea 100 extending between the anterior and posterior sides 102 and 104 of the artificial corneal 100. As the artificial cornea 100 is a mesoplant that connects the interior ocular environment with an exterior environment, better device fixation and biointegration helps provide a better seal that isolates the interior ocular environment from foreign media such as bacteria, virus, fungi or other microbes, which helps provide a higher probability of device retention and a lower probability of device extrusion.

In some examples, the tissue integration element 300 may be applied to the optical element 200 according to any known attachment method including, but not limited to adhesives, thermal bonding, pressure, or molding. In some examples, the polymer material is laser cut using a CO2 laser. Specifically, the radius of the cut for the inner diameter of the tissue integration element 300 to be situated adjacent to the anterior optical surface 210 is sized so that the placement of the tissue integration element 300 on the optical element 200 can be achieved without a significant gap between the tissue integration element 300 and the surface 224. In such examples, the tissue integration element 300 is placed with the cut hole concentric with the optical element in a lamination fixture. In order to shape the optical curvature of the anterior optical surface 210 and the posterior optical surface 214, lenses are used on the top and bottom of the optical element 200 and shape and lamination are achieved simultaneously by applying a pressure greater than five (5) psi to the lenses. Lamination of the tissue integration element 300 to the peripheral surface 208 is achieved in a similar fashion by cutting the outer diameter of the tissue integration element 300 after a first lamination of the tissue integration element 300 to the anterior surface 220, folding the polymeric material of the tissue integration element 300 onto the peripheral surface 208 and constraining the polymeric material of the tissue integration element 300 contacting the peripheral surface 208 radially around the peripheral surface 208. Thereafter, in some examples, the complete lamination assembly is placed in an oven for top and side lamination at 175° C. for 20 min each.

In various examples, the tissue integration element 300 is additionally or alternatively subjected to the one or more processes to modify the microstructure after being applied to the optical element 200. For example, after the tissue integration element 300 is applied to the optical element 200, the polymer material of the tissue integration element 300 and/or the optical element 200 may be subjected to one or more wetting processes (e.g., hydrophilic treatments) such that the polymer material of the tissue integration element 300 is wettable to ocular fluids. Such a configuration helps provide for cosmesis and rapid biointegration. In various examples, by being wettable, the tissue integration element 300 also becomes nearly transparent such that the artificial cornea 100 resembles the natural cornea in appearance. In some examples, an additional advantage of a wettable tissue integration skirt is that it provides for easier and faster ingress of ocular fluids and extracellular matrix. Such a configuration generally facilitates faster biointegration, which in turn, lowers the probability of infection and extrusion.

In some examples, the artificial cornea 100 may be subjected to one or more processes to achieve a desired shape. In some examples, these processes may achieve a desired shape that conforms to the shape of the penetration made in the patient's cornea. In some examples, these processes may achieve a desired shape and/or contour of one or more of the optical surfaces of the artificial cornea 100 (e.g., for proper light refraction). Such processes include the use of glass lenses made to a specific radius of curvature that is directly transferred to the optical element via a secondary molding procedure consistent with the description above. In other examples, a refractive surface is additionally or alternatively achieved through the use of machined surfaces using stainless steel or other suitable materials. In some examples, such surfaces could also be made to have special curvatures that offsets inherent optical distortions specific to the patient's eye.

As mentioned above, in some examples, the tissue integration element 300 is applied to the optical element 200 such that a portion of the anterior side 204 of the optical element 200 is covered or otherwise concealed by the tissue integration element 300. Specifically, in some examples, the tissue integration element 300 is applied at least to the anterior surface 220 of the annular flange 218. In some such examples, a thickness of the portion of the tissue integration element 300 applied to the anterior surface 220 of the annular flange 218 corresponds to the amount beyond which the anterior protrusion 212 protrudes beyond the anterior surface 220 of the annular flange 218. That is, in various examples, the tissue integration element 300 is applied to the anterior side 204 of the optical element 200 such that the anterior side 102 of the artificial cornea 100 is smooth. In such examples, a transition between the anterior optical surface 108 of the artificial cornea 100 and the portion of the tissue integration element 300 applied to the anterior side of the optical element 200 is smooth (e.g., free of protrusions, gaps, etc.). A smooth transition between the anterior optical surface 108 and the tissue integration element 300 provides that the anterior side 102 of the implanted artificial cornea 100 does not cause discomfort or irritation, or interfere with other portions of the patient's anatomy (e.g., such as the patient's eyelid). In addition, the incorporation of the tissue integration element 300 along a portion of the anterior side 102 of the artificial cornea 100 promotes a proliferation of tissue ingrowth along a portion of the anterior side 102 of the artificial cornea. It is to be appreciated that, while the tissue integration element 300 is shown in FIG. 4 as being applied across an entirety of the peripheral surface 208 of the annular flange 218, in some examples, the tissue integration element 300 may applied to a portion of less than all of the peripheral surface 208.

In some embodiments, the peripheral surface 208 may be stepped or otherwise partially recessed to accommodate the polymer material of the tissue integration element 300. In some embodiments, however, the polymer material of the tissue integration element 300 is applied to the peripheral surface 208 of the optical element such that the tissue integration element 300 extends from the anterior surface 220 to the posterior surface 222 of the annular flange 218. Such a configuration provides for tissue ingrowth about a periphery of the artificial cornea 100.

In some examples, the portion of polymer material of the tissue integration element 300 coupled to the anterior surface 220 of the annular flange 218 and the portion of the polymer material of the tissue integration element 300 coupled to the peripheral surface 208 of the optical element 200, together, form a single monolithic member. In some examples, the tissue integration element 300 may be pre-formed or pre-configured to mirror the relative orientations of surfaces of the optical element to which it is being attached or coupled. In other examples, the tissue integration element 300 is compliant in that it can be manipulated to conform to the relative orientations of surfaces of the optical element to which it is being attached or coupled as it is being attached or coupled.

In some embodiments, the tissue integration element 300 may be comprised of a plurality of discrete sections that are independently and separately coupled to the optical element 200. For example, a first section or portion of the tissue integration element 300 may be applied to the anterior surface 220 of the annular flange 218 while a second distinct section or portion of the tissue integration element 300 is applied to the peripheral surface 208 of the optical element 200. In some examples, these discrete sections or portions may be applied such that they abut or otherwise contact one another in a manner that facilitates a continuous coverage of the intended portions of the optical element 200. Thus, in some examples, a plurality of discrete sections of polymer material may be applied to the optical element 200 for form a tissue integration element 300 that is generally smooth and continuous.

It should be appreciated that the tissue integration element 300 may include or otherwise be comprised of multiple layers of the polymer material. In some examples, these layers may be oriented relative to one another to optimize one or more material properties of the tissue integration element 300, such as wettability, permeability, thickness, compliance, adhesion, transparency, etc. In some such examples, the various layers may be coupled together through one or more bonding, adhesion, or laminating processes as those of skill will appreciate.

In various examples, a surface condition of the portion of the tissue integration element 300 covering the anterior surface 220 of the annular flange 218 differs from a surface condition of the portion of the tissue integration element 300 covering the peripheral surface 208 of the optical element 200. Such a configuration operates to promote differing degrees and rates of tissue proliferation. For instance, in some examples, the portion of the tissue integration skirt coupled to the anterior surface 220 may be treated to promote rapid epithelialization and attachment thereto, while the portion of the tissue integration element 300 attached to the peripheral surface 208 may be treated to retard epithelial cell growth and promote stromal ingrowth.

In various embodiment, the tissue integration element 300 is applied to the optical element 200 such that the posterior side 206 of the optical element 200 remains uncovered or otherwise exposed. That is, in various examples, the posterior side 206 of the optical element 200 remains free from coverage by the tissue integration element 300. For example, as shown in FIG. 4, the tissue integration element 300 is applied to the optical element 200 such that the posterior side 104—including the posterior optical surface 214 and the posterior surface 222 of the annular flange 218—of the artificial cornea 100 is exposed or not otherwise covered by an anchoring material. Thus, in various examples, the tissue integration element 300 is applied to the optical element 200 such that the tissue integration element 300 does not otherwise contact the posterior side 206 of the optical element 200 including the posterior optical surface 214 and the posterior surface 222 of the annular flange 218. Put differently, in various examples, the tissue integration element 300 is applied to the optical element 200 such that the posterior side 206—including the posterior optical surface 214 and the posterior surface 222—remains free from contact with the tissue integration element 300 such that the posterior side 206 is otherwise exposed to the eye (e.g., eye interior and/or eye tissue bed). Disposing the tissue integration element 300 on the posterior optical surface 214 could inhibit light transmission and lead to optical fouling.

However, those of skill in the art should appreciate that, in various examples, the tissue integration element 300 may be partially or fully disposed across posterior surface 222.

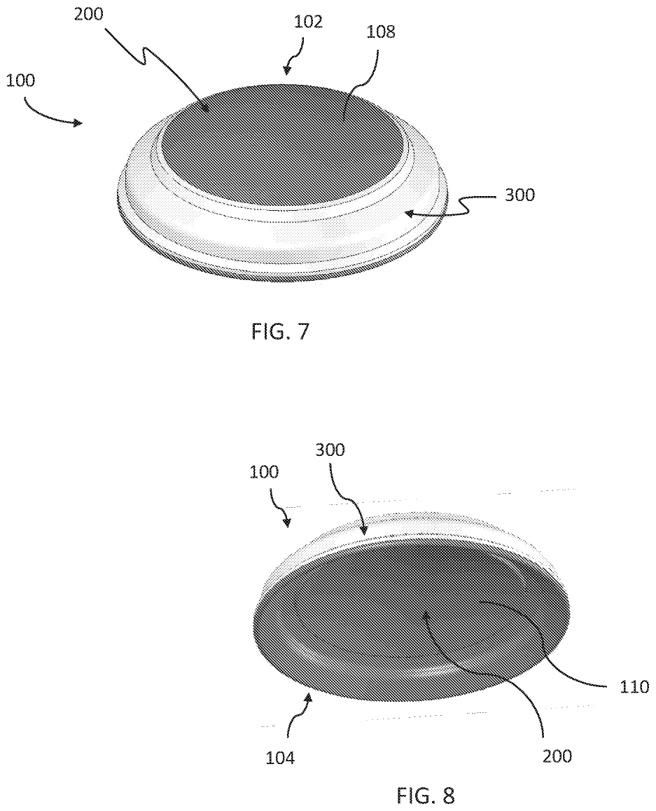

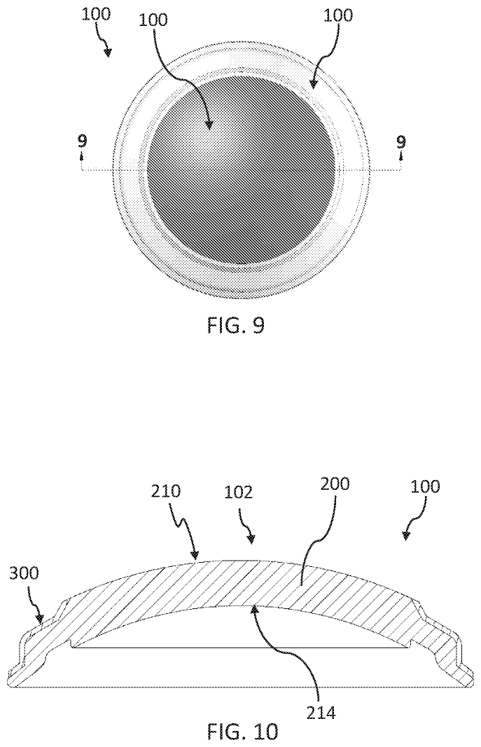

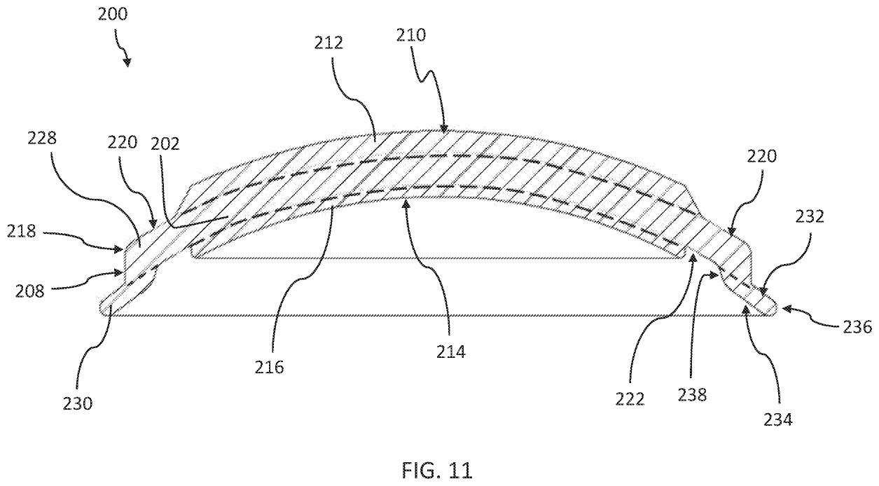

FIGS. 7 to 11 show an example of another artificial cornea 100. The artificial cornea 100 of FIGS. 7 to 11 includes an optical element 200 and a tissue integration element 300 coupled to the optical element 200. As shown in FIG. 11, the optical element 200 includes a body 202 similar to the body 202 discussed above with regard to FIG. 5 in that the body 202 includes anterior and posterior protrusions 212 and 216 defining anterior and posterior optical surfaces 210 and 214. The annular flange 218 of the body 202 shown in FIGS. 7 to 11 is different, however, from the annular flange of the body shown in FIGS. 1 to 5. In particular, as shown in FIG. 11, the annular flange 218 is defined by a first flange component 228 (FIG. 11) and a second flange component 230 (FIG. 11), each of which may additionally be described as flange portions, flange layers, flange segments, or flange features, for example. The first flange component 228 may be defined as a first annular portion of the body 202 of the optical element 200 extending radially outwardly of one or more of the anterior and posterior protrusions 212 and 216, while the second flange component 230 may be defined as a second annular portion of the body 202 of the optical element 200 extending radially outwardly of one or more of the anterior and posterior protrusions 212 and 216, and including a portion that extends radially outwardly of the first flange component 228. In various embodiments, the second flange component 230 is situated posteriorly of the first flange component 228.

In various embodiments, the first and second flange components 228 and 230 form a single monolithic body defining the annular flange 218. Thus, it is to be understood that the first and second flange components 228 and 230 may be integral with one another, although separate, connected parts are also contemplated. Similarly, the annular flange 218 may be integral with the other portions of the body 202 (e.g., the anterior and posterior protrusions 212 and 216) such that the first and second flange components 228 and 230 are integral with the anterior and posterior protrusions 212 and 216 to collectively define the body 202.

The first flange component 228 may include an anterior surface 220 and a peripheral surface 208, (e.g., in a similar manner to that discussed above with regard to the optical element of FIGS. 1 to 5). The second flange component 230 may include an anterior surface 232, a posterior surface 234, and a peripheral edge or surface 236 situated between or otherwise forming a transition between the anterior and posterior surfaces 232 and 234. In some embodiments, the peripheral edge 236 (also referred to as a peripheral surface 236) of the second flange component 230 and a peripheral edge (or outermost peripheral edge) of the optical element 200 are the same. In various embodiments, the first and second flange components 228 and 230 are oriented such that the anterior surface 220 of the first flange component 228 is situated anterior to the anterior surface 232 of the second flange component 230. Conversely, the posterior surface 234 of the second flange component 230 is situated posterior to the posterior surface 222 of the first flange component 228. In some embodiments, a transition 238 is defined between the posterior surfaces 222 and 234 of the first and second flange components 228 and 230, respectively. The transition 238 may be smooth and continuous, or alternatively may be stepped or discontinuous, as those of skill should appreciate. As shown in FIG. 11, the peripheral surface 236 of the second flange component 230 extends further radially outwardly than does the peripheral surface 208 of the first flange component 228. Additionally, as shown in FIG. 11, the posterior surface 222 of the first flange component 228 is situated anterior to each of the posterior surface 234 and the posterior optical surface 214 such that the posterior surface 222 defines an annular recess between the posterior surface 234 and the posterior optical surface 214.

As shown in FIG. 10, the tissue integration element 300 is coupled to the annular flange 218 such that a first portion of the tissue integration element 300 is coupled to the first flange component 228 and such that a second portion of the tissue integration element 300 is coupled to the second flange component 230. In particular, the tissue integration element 300 is coupled to each of the anterior and peripheral surfaces 220 and 208 of the first flange component 228 and to the anterior surface 232 of the second flange component 230. It should be appreciated, however, that the tissue integration element 300 may additionally be coupled to the peripheral surface 236 of the second flange component 230. As shown in FIG. 11, the tissue integration element 300 is not coupled to the posterior surfaces 222 and 234 of the first and second flange components, respectively.

The configuration of the body 202 illustrated in FIGS. 7 to 11 may be advantageous from several respects. For example, in accordance with some examples, the configuration of the body 202 provides additional surfaces for supporting the native corneal tissue of the eye after implantation of the artificial cornea 100, which helps with successful biointegration. For example, native tissue is permitted to grow into and onto the tissue integration element 300 where it is coupled to the anterior surface 220 and peripheral surface 208 of the first flange component 228, as well as where the tissue integration element 300 is coupled to the anterior surface 232 of the second flange component 230.

In various embodiments, the first flange component 228 of the annular flange 218 additionally operates as an element through which one or more fastening elements can be passed (e.g., one or more sutures) for initially securing the artificial cornea 100 to the tissue of the eye, as discussed further below.

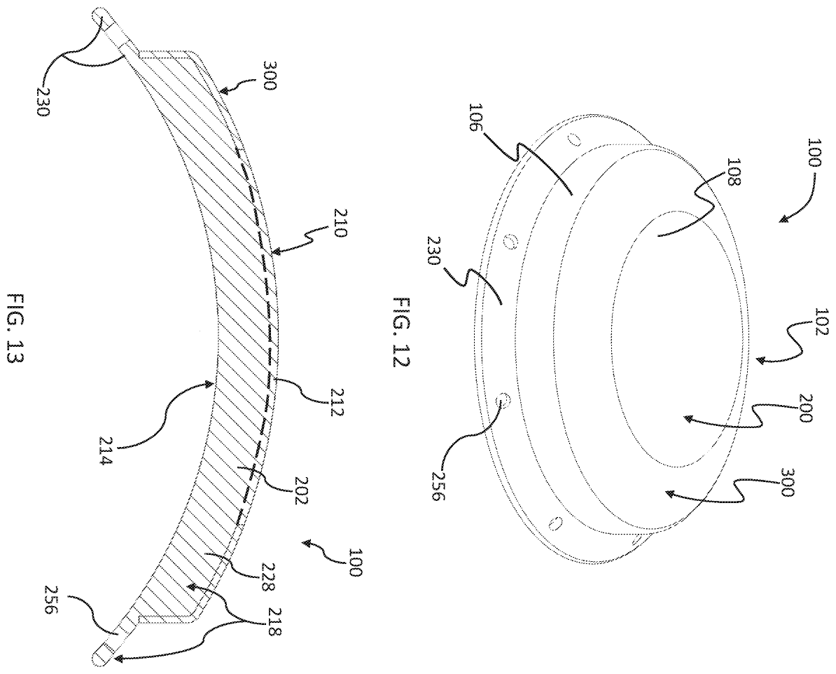

FIGS. 12 and 13 show an example of another artificial cornea 100. The artificial cornea 100 of FIGS. 12 and 13 includes an optical element 200 and a tissue integration element 300 coupled to the optical element 200. As shown in FIG. 13, the optical element includes a body dissimilar to the body 202 discussed above with regard to FIGS. 5, 10, and 11 in that while the body 202 shown in FIGS. 5, 10, and 11 includes an anterior protrusion 212 defining an anterior optical surface 210, the posterior optical surface 214 as depicted in FIG. 13 is not defined by an anterior protrusion. The annular flange 218 of the body 202 shown in FIGS. 12 and 13 is defined by a first flange component 228 (FIG. 13) and a second flange component 230 (FIG. 13), each of which may additionally be described as flange portions, flange layers, flange segments, or flange features, for example. The first flange component 228 is similar to the first flange component 228 discussed above in regards to FIGS. 7-11, and may be defined as a first annular portion of the body 202 of the optical element 202 extending radially outwardly from anterior protrusion 212. Second flange component 230 shown in FIGS. 12 and 13 is different, however, from the second flange component 230 of the body shown in FIGS. 7-11. In particular, as shown in FIGS. 12 and 13, the second flange 230 includes at least one aperture 256 passing from an anterior surface of the second flange 230 to a posterior surface of the second flange 230. The aperture 256 has a diameter of sufficient size to allow cells to grow, proliferate, or otherwise advance therethrough. Apertures 256 facilitate coupling of the artificial cornea 100 to the surrounding tissue of the eye. In some examples, apertures 256 are configured such that tissue growth can span (i.e., proliferate) through the second flange 230, which helps maintain a position of the artificial cornea 100 within the eye.

In various embodiments, the first and second flange components 228 and 230 form a single monolithic body defining the annular flange 218. Thus, it is to be understood that the first and second flange components 228 and 230 may be integral with one another, although separate, connected parts are also contemplated. Similarly, the annular flange 218 may be integral with the other portions of the body 202, although separate, connected parts are also contemplated. In some embodiments, second flange 230 comprises the same material as first flange 228 and/or body 202. In other embodiments, second flange 230 comprises a different material than first flange 228 and body 202. Apertures 256 may be formed in the second flange by micro drilling techniques such as, for example: mechanical micro drilling, such as ultrasonic drilling, powder blasting or abrasive water jet machining (AWJM); thermal micro drilling, such as laser machining; chemical micro drilling, including wet etching, deep reactive ion etching (DRIE) or plasma etching; and hybrid micro drilling techniques, such as spark-assisted chemical engraving (SACE), vibration-assisted micromachining, laser-induced plasma micromachining (LIPMM), and water-assisted micromachining. In other embodiments, second flange 230 comprises a different material than first flange 228 and body 202, and apertures 256 are a characteristic of the material of second flange 230. That is, the microstructure of the material itself of second flange 230 includes pores of sufficient size to form apertures 256. In certain embodiments, apertures 256 have a diameter of about 75 μm to about 600 μm. In particular embodiments, apertures 256 have a diameter of about 200 μm to about 300 μm. In a particular embodiment, apertures 256 have a diameter of about 300 μm.

The surfaces of the first and second flange components 228 and 230 of the artificial cornea 100 of FIGS. 12 and 13 are similar to those described above for the artificial cornea 100 of FIGS. 7-11.

As shown in FIG. 13, the tissue integration element 300 is coupled to the first flange component 228. In particular, the issue integration element 300 is coupled to each of the anterior and peripheral surfaces of the first flange component 228. In should be appreciated, however, that the tissue integration element 300 may additionally be coupled to one or both of the anterior and peripheral surfaces of the second flange component 230.

In some embodiments, an artificial cornea 100 similar to that shown in FIGS. 7-11, having an anterior protrusion 212, includes apertures 256 in second flange 230.

The configuration of the second flange component 230 as illustrated in FIGS. 12 and 13 may be advantageous in that it provides for tissue growth across the thickness of the artificial cornea, providing long-term mechanical anchoring of the artificial cornea. While in some embodiments tissue may grow into tissue integration element 300, having tissue growth across the thickness of the artificial cornea may provide a higher level of anchoring and retention.

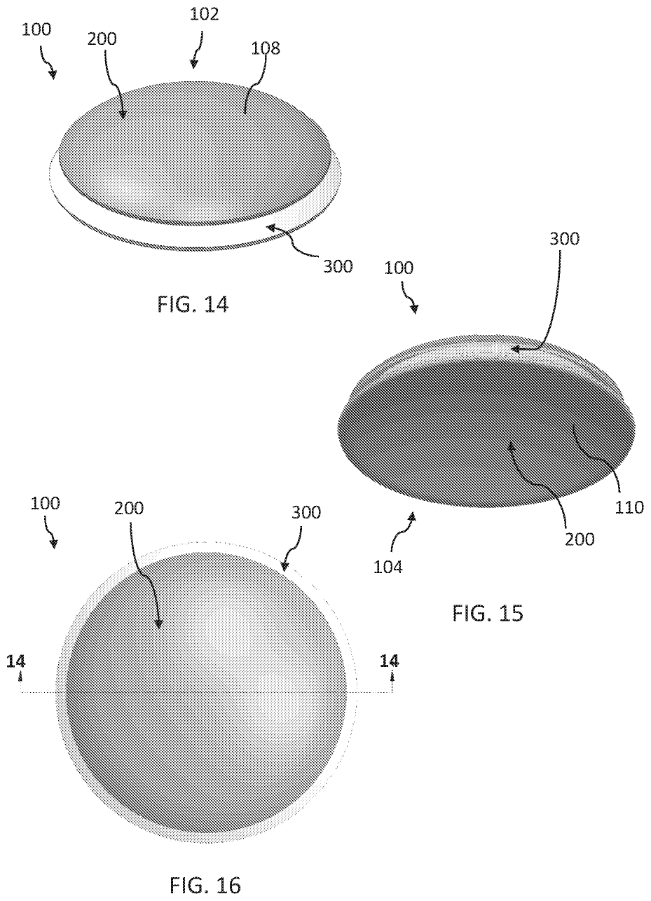

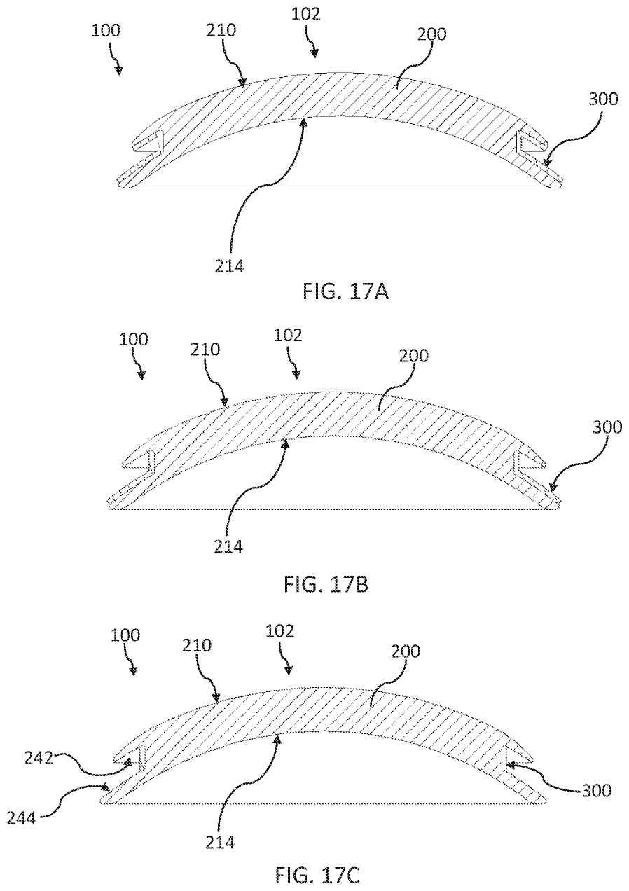

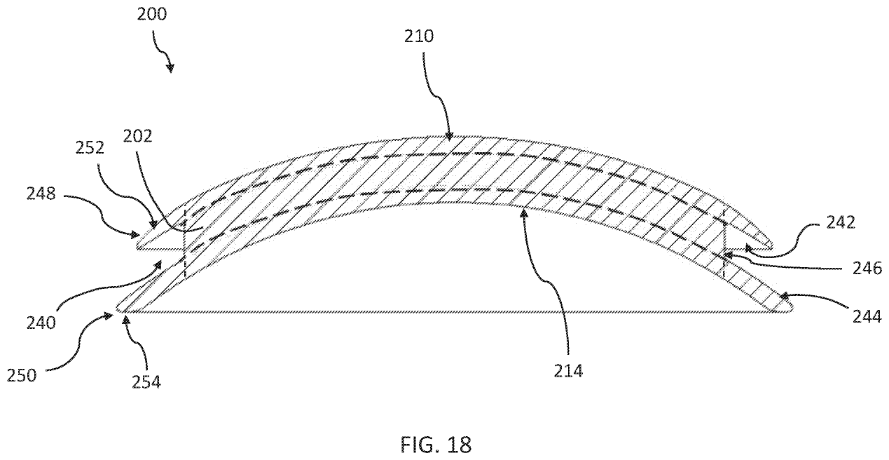

FIGS. 14 to 18 show an example of another artificial cornea 100. The artificial cornea 100 of FIGS. 14 to 18 includes an optical element 200 and a tissue integration element 300 coupled to the optical element 200. As shown in FIG. 18, the optical element 200 includes a body 202 that includes anterior and posterior optical surfaces 210 and 214. However, unlike the other configurations discussed herein, the body 202 does not include a radially extending annular flange to which a tissue integration element is coupled, but instead defines a peripherally extending recess 240 that is configured to accommodate native corneal tissue therein. The peripherally extending recess 240 is similarly configured to accommodate the tissue integration element 300, as shown in FIGS. 17A-17C. With continued reference to FIGS. 17A-17C and 18, the peripherally extending recess 240 is defined by a plurality of surfaces, including a first surface 242, a second surface 244 opposite the first surface 242, and a third surface 246 situated between and extending transverse to the first and second surfaces 242 and 244.

In some embodiments, the optical element 200 shown in FIG. 18 may be described as including a body 202 having anterior and posterior optical surfaces 210 and 214 and a plurality of flanges, including anterior flange 248 and posterior flange 250, each extending radially outwardly thereof and thereabout. The anterior and posterior flanges 248 and 250 are offset from one another along a longitudinal axis of the optical element 200 such that the peripherally extending recess 240 is defined therebetween. In some embodiments, the third surface 246 may be understood to correspond to a peripheral surface of the body 202, while the first surface 242 is understood to correspond to a posterior surface of the anterior flange 248, and while the second surface 244 is understood to correspond to an anterior surface of the posterior flange 250. In some embodiments, an anterior surface 252 of the anterior flange 248 and the anterior optical surface 210, collectively, define the anterior side of the optical element 200 shown in FIGS. 14 to 18. Similarly, in some embodiments, a posterior surface 254 of the posterior flange 250 and the posterior optical surface 214, collectively, define the posterior side of the optical element 200 shown in FIGS. 14 to 18.

As shown in FIG. 17A, the tissue integration element 300 is coupled to each of the first, second, and third surfaces 242, 244, and 246 such that, once implanted in a patient's eye, corneal or other associated eye tissue is permitted to grow into the tissue integration element 300 along each of the first, second, and third surfaces 242, 244, and 246.

As shown in FIG. 17B, the tissue integration element 300 is coupled to the second and third surfaces 244 and 246 such that, once implanted in a patient's eye, corneal or other associated eye tissue is permitted to grow into the tissue integration element 300 along each of the second and third surfaces 244 and 246, while first surface 242, which in such a configuration is resistant to tissue ingrowth, serves as an alignment edge, aligning the artificial cornea within the patient's eye.

As shown in FIG. 17C, the tissue integration element 300 is coupled only to third surface 246 such that, once implanted in a patient's eye, corneal or other associated eye tissue is permitted to grow into the tissue integration element 300 along third surface, while first and second surfaces 242 and 244, which in such a configuration are resistant to tissue ingrowth, serve as alignment edges, aligning the artificial cornea within the patient's eye.

In various examples, the artificial cornea illustrated and described herein is implanted in conjunction with a penetrating keratoplasty surgical procedure wherein a full-thickness section of tissue is removed from the diseased or injured cornea using a surgical cutting instrument, such as a trephine or a laser. In various examples, a circular full-thickness plug of the diseased or damaged cornea is removed, leaving a tissue bed of corneal tissue to which the artificial cornea 100 can be affixed. In such a configuration, a portion of or all of the posterior side 104 of the artificial cornea 100 is suspended above the interior of the eye. That is, a portion of or all of the posterior side 104 of the artificial cornea 100 is not supported by the existing corneal tissue of the eye. In cases involving a full thickness excision of the cornea, the cornea is generally removed from epithelium to endothelium.

The artificial cornea 100 illustrated and described herein is also configured to be implanted in conjunction with partial thickness surgical procedures, such as Descemet's Stripping and Automated Endothelial Keratoplasty (DSAEK), where less than a full-thickness section of tissue is removed from the diseased or injured cornea, leaving the residual bed of healthy cornea tissue. In these embodiments, the diseased portion of the anterior cornea is excised and the artificial cornea 100 is positioned on the residual bed of healthy cornea tissue.

In various examples, the artificial cornea discussed herein is configured such that it can be temporarily folded and deformed to help facilitate implantation. That is, unlike many conventional designs, the artificial cornea (e.g., including the body 202 of the optical element 200) is configured to be compliant and non-rigid. For instance, during an implantation procedure, a physician may need to fold or deform the artificial cornea to achieve a proper orientation and/or to properly seat the artificial cornea in the native tissue bed. In some instances, an independent constraining member may be utilized to temporarily maintain a deformation of the artificial cornea prior to and during the implantation procedure. In various examples, the artificial cornea 100 is sufficiently resilient to assume an undeformed geometry upon being released into or onto the tissue bed and/or being secured to the tissue bed. Configuring the artificial cornea such that it is compliant and non-rigid also provides that the intra-ocular pressure of the eye can be monitored according to conventional methods while the artificial cornea is implanted.

For instance, because the artificial cornea is compliant (e.g., has a measure of compliance comparable to that of a native cornea), the intra-ocular pressure of the eye in which the artificial cornea is implanted can be determined through known ocular tonometry methods, including but not limited to, applanation tonometry, goldmann tonometry, dynamic contour tonometry, electronic indentation tonometry, rebound tonometry, pneumatonometry, indentation or impression tonometry, and non-contact tonometry. When used in combination with the artificial cornea discussed herein, these methodologies involve measuring a deformation response along an interface between the artificial cornea and the native cornea tissue when acted on by a force external to the eye. For instance, measurement may occur at one or more locations along a perimeter of the optical element where the optical element and the native corneal tissue (e.g., the corneal limbus) interest or interface. This may include purely native corneal tissue, or may include a region where the native corneal tissue overlaps the artificial cornea. The external force delivered by the tonometry equipment may include air pressure, and/or may include an external force that is applied by a physical body contacting the measurement region of the eye. Other methodologies exist for measuring intra-ocular pressure via interactions with other regions of the eye (e.g., the sclera), and are to be understood as being distinct from ocular tonometry involving measuring intra-ocular pressure along an interface between the artificial cornea and the native corneal tissue. It is to be appreciated that ocular tonometry is not possible for conventional rigid artificial cornea designs, as rigid conventional artificial cornea designs are not themselves sufficiently deformable nor is the interface along the native eye tissue and such conventional rigid artificial cornea designs.

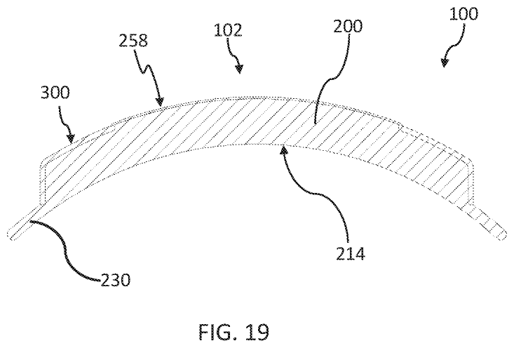

FIG. 19 shows an embodiment of an artificial cornea 100. The artificial cornea 100 of FIG. 19 is similar to that of FIG. 13, although with two key distinctions. Firstly, as shown, second flange component 230 of FIG. 19 does not include apertures 256. Secondly, the embodiment shown in FIG. 19 further includes corneal epithelial cell growth layer 258. Corneal epithelial cell growth layer 258 is configured to encourage and support formation and maintenance of an organized monolayer of corneal epithelial cells across the anterior side 204 of the optical element 200. Corneal epithelial cell growth layer 258 is deposited on the anterior side 204 of the optical element 200 such that the anterior side 102 of the artificial cornea 100 is smooth. In such examples, a transition between the corneal epithelial cell growth layer 258 and the portion of the tissue integration element 300 applied to the anterior side of the optical element 200 is smooth (e.g., free of protrusions, gaps, etc.). A smooth transition between the epithelial cell growth layer 258 and the tissue integration element 300 provides that the anterior side 102 of the implanted artificial cornea 100 does not cause discomfort or irritation, or interfere with other portions of the patient's anatomy (e.g., such as the patient's eyelid). In addition, while tissue integration element 300 promotes a proliferation of tissue ingrowth along a portion of the anterior side 102 of the artificial cornea, epithelial cell growth layer 258 promotes the formation of an organized monolayer of corneal epithelial cells over the anterior side 204 of the optical element 200. It is to be appreciated that, while the tissue integration element 300 is shown in FIG. 19 as being applied across an entirety of the peripheral surface 208 of the annular flange 218, in some examples, the tissue integration element 300 may applied to a portion of less than all of the peripheral surface 208. Similarly, while epithelial cell growth layer 258 is shown in FIG. 19 as being applied across the entirety of the anterior side 204 of the optical element 200 not covered by tissue integration element 300, in some examples, the epithelial cell growth layer 258 may be applied to a portion of less than all of the anterior side 204 of the optical element 200 not covered by tissue integration element 300. Though described in association with the examples above, epithelial cell growth layer 258 can be incorporated into any example of artificial cornea disclosed and described herein.

In some embodiments, epithelial cell growth layer 258 includes one or more plasma coatings (positively or negatively charged), glycoproteins, collagens and gelatins, and/or proteoglycans. Useful glycoproteins include, for example, fibronectin, laminin, and vitronectin. Useful collagen types include, for example, type I, type II, type III, type IV, and type V. Useful proteoglycans include, for example, versican, perlecan, neurocan, aggrecan, and brevican. In certain embodiments, the epithelial cell growth layer includes a mixture of molecules, forming a matrix. The composition of the epithelial cell growth layer 258 is selected such that an organized monolayer of corneal epithelial cells is encouraged to grow and proliferate across the anterior side 204 of the optical element 200.

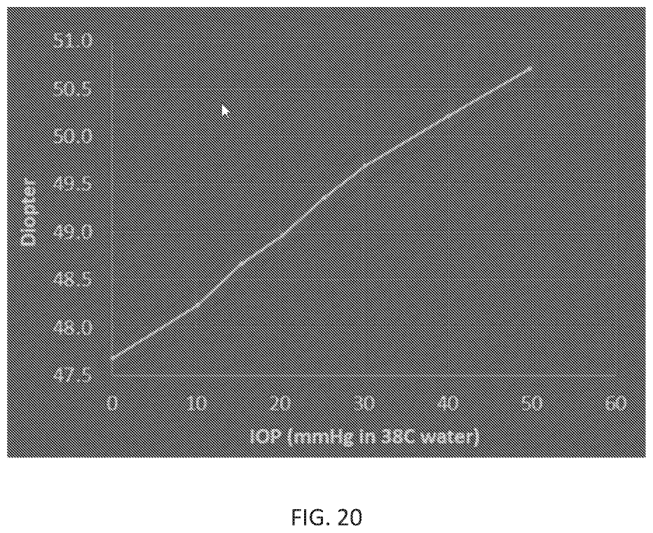

Turning now to FIG. 20, a graphical representation of the experimental relationship between diopter and intra-ocular pressure determined according to the ocular tonometry methods discussed above when the artificial cornea discussed herein is implanted. A healthy intra-ocular pressure within the eye is within a range of between ten (10) and twenty (20) millimeters of mercury (mmHg). As shown, the artificial cornea when implanted is associated with a diopter of approximately 48.2 diopter at 10.1 millimeters of mercury (mmHg), and a diopter of approximately 49 diopter at 20.3 millimeters of mercury (mmHg). The slope of the line in FIG. 20 corresponds to the conformability or elasticity of the measured region, which is represented in units of diopter per millimeters of mercury (mmHg). The graph shown in FIG. 20 illustrates the conformability of the interface region between the artificial cornea and the native corneal tissue, which is approximately 0.064 diopter per millimeter of mercury. The conformability of the interface region is based, at least in part, on the conformability of the native corneal tissue and the artificial cornea material located at and surrounding the measured interface region. Thus, it is to be appreciated that the artificial cornea discussed herein is of a sufficient conformability to facilitate an accurate ocular tonometry measurement at the interface region that is not otherwise achievable with conventional rigid artificial cornea designs.

In some embodiments, a compliant or elastic artificial cornea may have a conformability or elasticity greater than zero and up to approximately 0.075 diopter per millimeter of mercury. Rigidity of an artificial cornea is understood to increase as the diopter/mmHg slope decreases, and rigidity of an artificial cornea is understood to decrease as the diopter/mmHg slope increases. Accordingly, although not illustrated in FIG. 20, as slope approaches zero (0) diopter per millimeter of mercury (mmHg), a corresponding artificial cornea would approach minimal or zero elasticity, which is an elasticity that is consistent with many conventional artificial cornea designs. Consequently, an artificial cornea associated with a slope approaching zero (0) diopter per millimeter of mercury (mmHg) in a range of at least between ten (10) and twenty (20) millimeters of mercury (mmHg) results in poor accuracy in measuring intra-ocular pressure via interactions with the rigid artificial cornea, as the artificial cornea nor the native corneal tissue adjacent the rigid artificial cornea is not sufficiently deformable under testing conditions to accurately measure intra-ocular pressure.

Conversely, a slope increasing beyond 0.075 diopter per millimeter of mercury (mmHg) in a range of at least between ten (10) and twenty (20) millimeters of mercury (mmHg) becomes increasingly susceptible to perceptible vision changes during the course of the expected normal daily fluctuations in intra-ocular pressure in healthy patients. For instance, if a patient's intra-ocular pressure is expected to fluctuate between ten (10) and fifteen (15) millimeters of mercury (mmHg) during the course of a day, an artificial cornea having a compliance or elasticity of 0.075 diopter per millimeter of mercury (mmHg) would be expected to experience a vision differential of approximately 0.375 diopter. Comparatively, an artificial cornea having a compliance or elasticity of 0.05 diopter per millimeter of mercury (mmHg) would be expected to experience a vision differential of approximately 0.25 diopter under the same conditions, whereas an artificial cornea having a compliance or elasticity of 0.095 diopter per millimeter of mercury (mmHg) would be expected to experience a vision differential of approximately 0.475 diopter under the same conditions.

It is desirable to provide a sufficiently compliant artificial cornea while minimizing the potential for perceptible vision changes under expected intra-ocular pressure fluctuations. Accordingly, it is to be appreciated that the compliance or elasticity of the artificial cornea should be selected based on expected intra-ocular pressure fluctuations for the patient.

In some examples, a surgical implantation method requires undersizing the trephinated hole made in the host cornea relative to the diameter of the artificial cornea. In some examples, this is to account for the amount by which the excised host cornea grows when it experiences trauma (e.g., an incision). In some examples, such undersizing also operates to account for retraction due to partial corneal melting, post-surgery. In addition, such undersizing allows the wound to be air and liquid tight after suturing, which helps avoid infection risks due to ingress of pathogens.

In various examples, after the artificial cornea is properly positioned and oriented within the tissue bed of the existing corneal tissue, the artificial cornea is mechanically coupled to the existing corneal tissue. In various examples, one or more sutures are utilized to mechanically fasten the artificial cornea to the existing corneal tissue. In some other examples, an ophthalmic glue may additionally or alternatively be utilized for mechanically coupling the artificial cornea to the existing corneal tissue. In the case of suturing, the particular surgical suturing technique (e.g., interrupted, uninterrupted, combined, single, double, etc.) may vary based on a number of surgical indications as will be appreciated by those of skill in the art. In various examples involving the fastening of the artificial cornea to the existing corneal tissue by way of one or more sutures, the sutures generally extend into the annular flange 218 of the optical element 200 of the artificial cornea 100. In some examples, one or more sutures extend through only a portion of the annular flange 218. For example, one or more sutures may enter the anterior side 102 of the artificial cornea 100 and exit the artificial cornea 100 through the peripheral surface 208 and any tissue integration skirt material covering the peripheral surface 208 before entering the existing corneal tissue. In some examples, one or more sutures additionally or alternatively extend entirely through the annular flange 218 (including one or more of the first and second flange components 228 and 230). For example, one or more sutures enter the anterior side 102 of the artificial cornea 100 and exit the posterior surface 222 of the annular flange 218 before entering the existing corneal tissue. In one such example, the suture exiting the posterior surface 222 of the annular flange 218 may enter existing corneal tissue upon which the posterior surface 222 of the annular flange 218 is resting.

Those of skill should appreciate that one or more sutures may additionally or alternatively enter the annular flange through the peripheral surface 208 of the annular flange and any tissue integration skirt material covering the peripheral surface 208 and subsequently exit through the peripheral surface 208 of the annular flange and any tissue integration skirt material covering the peripheral surface 208. Additionally or alternatively, in some examples, one or more sutures may enter the annular flange through the peripheral surface 208 of the annular flange and any tissue integration skirt material covering the peripheral surface 208 and subsequently exit the posterior surface 222 of the annular flange 218. Those of skill should also appreciate that mechanically fastening or affixing (e.g., suturing) of the artificial cornea 100 to the existing corneal tissue may be temporary or permanent. For instance, in some examples, sutures provide mechanical fastening of the device after the implantation procedure, but subsequent tissue ingrowth into the tissue integration element 300 operates as a permanent mechanism for attachment.

In various embodiments, fastening the artificial cornea 100 to the existing corneal tissue operates to maintain a relative position between the artificial cornea 100 and the existing corneal tissue while corneal tissue grows into the tissue integration element 300, as those of skill will appreciate. Likewise, as those of skill will appreciate, fastening the artificial cornea 100 to the existing corneal tissue operates to maintain contact between the existing corneal tissue and the artificial cornea 100 while corneal tissue grows into the tissue integration element 300. Such a configuration also operates to seal the interior of the eye from the outside environment and potential ingress of bacteria.

In various examples, the sutures may comprise any suitable biocompatible material including nylon, polypropylene, silk, polyester and fluoropolymers such as ePTFE and other copolymers discussed herein.

While above-discussed embodiments include configurations where the skirt covers only a portion of the anterior surface, in some examples, the skirt may cover the entire anterior side including the anterior optical surface. Such a configuration helps facilitate the proliferation and integration of epithelial tissue across the entire anterior surface of the artificial cornea that is exposed to the external environment, which would help further biointegration. Additionally, such a configuration would increase optic wettability, and help minimize fouling. However, in certain cases, epithelial tissue growth across the entire anterior surface of the artificial cornea may be undesirable. For example, in certain instances, diseased tissue lacks the appropriate morphology to be a clear refracting surface. In such instances, the regenerated epithelium tissue is therefore unclear and could lead to optical fouling and should be avoided.

The inventive scope of this application has been described above both generically and with regard to specific examples. It will be apparent to those skilled in the art that various modifications and variations can be made in the examples without departing from the scope of the disclosure. Likewise, the various components discussed in the examples discussed herein are combinable. Thus, it is intended that the examples cover the modifications and variations of the inventive scope.

1. An artificial cornea comprising:
an optical element comprising a body having an anterior side and a posterior side, an annular flange extending about the body, the anterior side including an anterior optical surface and the posterior side of the body including a posterior optical surface; and
a tissue integration skirt coupled to the optical element, the tissue integration skirt being configured to promote tissue ingrowth, the tissue integration skirt being coupled to the optical element such that at least a portion of a periphery of the annular flange defined between the anterior and posterior sides of the optical element is covered by the tissue integration skirt.

2. The artificial cornea of claim 1, wherein the annular flange includes a first flange component and a second flange component situated posterior to the first flange component, the first flange component defining a first anterior surface and a peripheral surface, the second flange component defining a second anterior surface offset from the first anterior surface by the peripheral surface.

3. The artificial cornea of claim 2, wherein the tissue integration skirt is coupled to each of the first anterior surface, the peripheral surface, and the second anterior surface.

4. The artificial cornea of claim 2, wherein the first and second anterior surfaces of the annular flange are nonparallel.

5. The artificial cornea of claim 2, wherein the annual flange has a nonuniform thickness.

6. The artificial cornea of claim 2, wherein the first flange component and the second flange component each extend about the body radially outwardly therefrom.

7. The artificial cornea of claim 2, wherein the second flange component extents more radially outwardly than the first flange component.

8. The artificial cornea of claim 2, wherein the second flange comprises at least one aperture configured to allow tissue to proliferate therethrough.

9. The artificial cornea of claim 8, wherein the at least one aperture is formed by micro drilling.

10. The artificial cornea of claim 8, wherein the second flange comprises a material having a microstructure that forms the at least one aperture.

11. The artificial cornea of claim 1, wherein the posterior optical surface is offset from a posterior surface of the annular flange.

12. The artificial cornea of claim 11, wherein the offset between the posterior optical surface and the posterior surface of the annular flange is configured as a barrier to help resist a proliferation of tissue across the posterior optical surface.

13. The artificial cornea of claim 1, wherein the posterior side of the body is free from coverage by the tissue integration skirt.

14. The artificial cornea of claim 1, wherein the tissue integration skirt covers a portion of the anterior side of the optical element.

15. An artificial cornea comprising:
an optical element configured to resist tissue ingrowth, the optical element comprising a body having an anterior side and a posterior side, the anterior side including an anterior optical surface and the posterior side of the body including a posterior optical surface,
an annular flange extending about the body, the annular flange including a first flange component and second flange component situated posterior to the first flange component such that a peripheral surface of the body is defined between the first and second flange components, the first flange component defining a posterior flange surface, the second flange component defining an anterior flange surface offset from the posterior flange surface by the peripheral surface, and
a tissue integration skirt being configured to perm it tissue ingrowth, the tissue integration skirt being coupled to the peripheral surface.

16. The artificial cornea of claim 15, wherein the integration skirt is further coupled to the anterior flange surface, the posterior flange surface, or both the anterior flange surface and posterior flange surface.

17. The artificial cornea of claim 1, wherein the anterior optical surface is convex.

18. The artificial cornea of claim 1, wherein the posterior optical surface is concave.

19. The artificial cornea of claim 1, wherein the optical element comprises a fluoropolymer.

20. The artificial cornea of claim 19, wherein the fluoropolymer has been treated to render it hydrophilic.

21. The artificial cornea of claim 20, wherein the fluoropolymer is hydrophilic.

22. The artificial cornea of claim 1, wherein the optical element comprises a copolymer of tetrafluoroethylene (TFE) and perfluoromethyl vinyl ether (PMVE).

23. The artificial cornea of claim 1, wherein the artificial cornea is foldable.

24. The artificial cornea of claim 1, wherein the artificial cornea is configured such that an intra-ocular pressure of an eye can be measured in situ through ocular tonometry involving interactions with the artificial cornea.

25. The artificial cornea of claim 24, wherein the artificial cornea is configured such that an intra-ocular pressure of an eye can be measured in situ by measuring a deformation response of a region of the eye where the artificial cornea interfaces with native corneal tissue when acted on directly by a force external to the eye.

26. The artificial cornea of claim 25, wherein the external force is applied by a physical body contacting the measured interface region.

27. The artificial cornea of claim 1, wherein a refractive index of the artificial cornea is in a range of between 1.3 to 1.4.

28. The artificial cornea of claim 1, wherein the optical element is configured to resist tissue ingrowth.

29. The artificial cornea of claim 1, wherein the anterior optical surface is configured to permit tissue attachment thereto while resisting tissue ingrowth.

30. The artificial cornea of any one of claim 29, wherein the anterior optical surface includes a microstructure configured to permit tissue attachment to the anterior optical surface while resisting tissue ingrowth.

31. The artificial cornea of claim 29, wherein the anterior optical surface is at least partially covered by a corneal epithelial growth layer, the corneal epithelial growth layer being configured to encourage and support formation and maintenance of an organized monolayer of corneal epithelial cells over the anterior optical surface.

32. The artificial cornea of claim 1, wherein the optical element is formed of a material having a microstructure that is configured to resist tissue ingrowth.

33. The artificial cornea of claim 1, wherein the optical element is coated with a material that is configured to resist tissue ingrowth.

34. The artificial cornea of claim 1, wherein the tissue integration skirt is formed of a material having a microstructure that is configured to permit tissue ingrowth.

35. A method of forming an artificial cornea, the method comprising:
providing an optical element having an anterior side and a posterior side, an annular flange extending about the body, the posterior side of the body including a posterior optical surface,
providing a tissue integration skirt, the tissue integration skirt being configured to promote tissue ingrowth,
coupling the tissue integration skirt to the optical element such that a portion of a periphery of the annular flange defined between the anterior and posterior sides of the optical element is covered by the tissue integration skirt.

36. The method of claim 35, wherein the posterior optical surface is longitudinally offset from a posterior surface of the annular flange.

37. The method of claim 35, wherein the tissue integration skirt is further coupled to the optical element such that a portion of the anterior side of the optical element is covered by the tissue integration skirt.

38. The method of claim 35, wherein the optical element is configured to resist tissue ingrowth, and wherein the anterior side of the of the optical element is configured to permit tissue attachment while resisting tissue ingrowth.

39. A method of implanting an artificial cornea, the method comprising:
providing the artificial cornea of claim 1;
removing a section of corneal tissue from a patient's cornea to form a tissue bed of existing corneal tissue to which the artificial cornea can be affixed;
implanting the artificial cornea such that the posterior side of the artificial cornea is suspended above the interior of the eye; and
mechanically affixing the implanted artificial cornea to the existing corneal tissue of the tissue bed.

40. The method of claim 39, wherein removing a section of corneal tissue includes removing a full-thickness section of corneal tissue from the patient's cornea, and wherein implanting the artificial cornea includes implanting the artificial cornea such that the posterior side of the artificial cornea is unsupported by the existing corneal tissue of the tissue bed.
